(19) United States
(12) Patent Application Publication
(10) Pub. No.: US 2011/0087124 A1
(43) Pub. Date: Apr. 14, 2011

(54) Spirometer Toy

(76) Inventors: Patricia Bohman, Glenwood Spring, CO (US); Kelsey Bohman, Glenwood Springs, CO (US)

(21) Appl. No.: 12/972,456

(22) Filed: Dec. 18, 2010

Related U.S. Application Data
(62) Division of application No. 12/261,007, filed on Oct. 29, 2008.
Application No. 12/261,007 is a continuation-in-part of application No. 11/426,189, filed on Jun. 23, 2006, now abandoned.
(60) Provisional application No. 60/693,652, filed on Jun. 23, 2005.

(51) Int. Cl. A61B 5/08; F41B 1/00

(52) U.S. Cl. 600/538; 124/62

(57) ABSTRACT

The invention provides a toy that functions to propel a projectile through a tube at someone or something by exerting sufficient air pressure from the user's lungs through the tube. This action has the effect of challenging and building the user's respiratory system, including the respiratory musculature and lung capacity, through the repeated use of the toy.

### CROSS REFERENCE TO RELATED APPLICATIONS

This application is a division of U.S. patent application Ser. No. 12/261,007, filed Oct. 29, 2008, which is a continuation-in-part of U.S. patent application Ser. No. 11/426,189, filed Jun. 23, 2006, which claims the benefit of priority under 35 U.S.C. §119(e) to U.S. Provisional Patent Application Ser. No. 60/693,652 filed Jun. 23, 2005, each of which is incorporated herein, in its entirety, by this reference.

### TECHNICAL FIELD

The invention relates to an apparatus with which a user may propel projectiles using force created by air pressure from the user's lungs.

### BACKGROUND OF INVENTION

Spirometers are devices used to measure the volume and flow rate of gas exhaled by a user or patient. These measurements are important for physiological studies and for diagnostic analysis of the pulmonary performance of the patient. For example, exhaled volume and flow rate are often measured for people who suffer from asthma, bronchitis, mucoviscidosis, or who have had lung transplants. Especially for patients suffering from asthma, these data are used to make estimates of asthma medicine dosages and to anticipate an asthma crisis.

Spirometers are also known to have therapeutic effects in that their frequent and continual use strengthens the diaphragm, intercostal muscles and other muscles used to exhale as well as building lung capacity. Unfortunately, it is difficult to motivate the regular use of these devices to the extent necessary to develop the muscles and lung capacity that helps to overcome asthmatic attack or rehabilitate patients suffering respiratory illness, particularly amongst adolescents.

Thus, an apparatus capable of stimulating the user's curiosity and playfulness while aiding the development of lung capacity and respiratory musculature is desired.

### SUMMARY OF INVENTION

The present invention provides devices capable of holding the user's attention as a toy while strengthening the muscles of the user's respiratory system through repetitive use. One embodiment is an apparatus having a tube with a mouthpiece on one end and at least one closable aperture along the length of the tube. The tube is preferably a circular tube with a length between about 4 inches and about 36 inches and a diameter between about 5 mm and about 20 mm. The tube may be composed of wood, plastic, polymer, laminate, resin, metal, polycarbonate and combinations thereof. The aperture in the tube is preferably between about 10 mm and about 25 mm in diameter.

In one embodiment, the apparatus includes a closure for the aperture such as a sleeve, a door or an aperture tube. In a preferred embodiment, the closure is an aperture tube slidably engaged with, and disposed within the tube. In a similar embodiment, the closure is a sleeve tube slidably engaged with, and disposed around the tube. The closure may also be a door that slides over the aperture in the tube.

In a preferred embodiment, the apparatus includes a mouthpiece formed from the crimped end of the tube. Alternatively, the mouthpiece may be a material that is removably-engaged from the tube.

In one embodiment, the tube includes one or more projections forming a handle, a sight, a catch, a holder of additional projectiles, a means of measuring and/or recording air pressure exerted through the mouthpiece and/or a means of adjusting resistance to the propulsion of a projectile from the tube. In a related embodiment, the mouthpiece may contain one or more projections forming a handle, a sight, a catch, a holder of additional projectiles, a means of measuring and/or recording the air pressure exerted through the mouthpiece and/or a means of adjusting the resistance to the propulsion of a projectile from the tube.

The apparatus may be configured to accept and to propel a projectile selected from the group consisting of a marshmallow, a mini-marshmallow, a pea, a wad of paper, a wad of gum, a ball and a piece of foam.

A related embodiment provides a kit containing the apparatus and at least one projectile such as a marshmallow, a mini-marshmallow, a pea, a wad of paper, a wad of gum, a ball and a piece of foam.

Another embodiment provides a method of enhancing the respiratory system in a user comprising inserting a projectile through the aperture and into the tube of the apparatus, placing the lips of the user on the mouthpiece of the apparatus, and exhaling through the mouthpiece with sufficient force to expel the projectile from the tube.

### BRIEF DESCRIPTION OF DRAWINGS

FIG. 1 shows one specific embodiment of the present invention fully assembled.

FIG. 2 shows the embodiment of FIG. 1, having an inner aperture tube removed from the tube.

FIG. 3 shows one specific embodiment of the present invention in which a crimped mouthpiece is provided at one end of the tube and a handle is provided as one projection from the tube.

FIG. 4 shows a more detailed image of the embodiment shown in FIG. 1 in which the apertures in the tube and the inner aperture tube have been aligned length-wise in the tube but are offset to effectively seal the tube.

FIG. 5 shows another specific embodiment of the present invention fully assembled.

FIG. 6 shows the embodiment of FIG. 5, having an inner aperture tube removed from the tube.

FIG. 7 shows another specific embodiment of the present invention having an outer sleeve tube and no aperture tube.

FIG. 8 shows the top view of the embodiment of FIG. 7.

FIG. 9 shows a plan view of a specific embodiment of the present invention.

FIG. 10 shows a top view of the embodiment shown in FIG. 9.

FIG. 11 shows a side view of the embodiment shown in FIG. 9.

FIG. 12 shows a view into the mouthpiece of the embodiment shown in FIG. 9.

### DESCRIPTION OF EMBODIMENTS

The present invention is drawn to an apparatus that functions to enhance the development or rehabilitation of a user's respiratory function while holding the user's interest by the provision of a means to propel projectiles at a distance.

The apparatus may include a tube having a closable aperture and, optionally, a crimped end-piece that functions as a mouthpiece. The aperture includes a closure that allows the insertion and positioning of a projectile within the tube. In practice, the user inserts a projectile into the cylindrical tube through the aperture and secures the closure before putting the mouthpiece end between the user's lips. The user then exhales into the tube through the mouthpiece to propel the projectile out of the end of cylindrical tube opposite the mouthpiece.

The tube may be of any length, but the typical length is between about 4 inches and about 36 inches. Preferably, the length of the tube is about 8 inches or about 10 inches or about 12 inches or about 14 inches or about 16 inches or about 18 inches or about 20 inches. Most preferably, the length of the tube is about 9 inches.

The cross-section of the tube may have any closed shape, but a circular or nearly-circular cross-section is the preferred shape. Thus, a preferred embodiment is a cylindrical tube having an internal diameter between about 5 mm and about 20 mm. Most preferably, the internal diameter is about 13 mm. Similarly the external diameter of the cylindrical tube is between about 8 mm and about 30 mm and will vary depending upon the internal diameter and the characteristics of the material from which the tube is composed. Preferably, the external diameter of the tube is about 15 mm.

The tube may be composed of any suitably sturdy material to withstand repeated use by a human user without significant and rapid deterioration. This material should therefore be resistant or at least partially resistant to moisture and the contents of expectorated mucus and saliva that may accompany gases exhaled into the apparatus. Preferably, the tube is composed of a rigid material although, in specific embodiments, the tube may be flexible in order to adjust the distance or direction of travel of a projectile and to adjust the resistance that the user must overcome to expel a projectile from the tube. The tube may be composed of wood, plastic, polymer(s), laminates, resins, metals, combinations of these materials and the like. Preferably, the tube is composed of clear or translucent material(s) such that the user can judge the placement and movement of a projectile placed in the tube. Most preferably, the tube is composed of extruded polycarbonate which can be made in many colors. The tube and any projections or attachments to the tube may be produced by any standard technique such as injection molding or extrusion.

The aperture in the tube may be of any size suitable to allow easy placement of a projectile within the tube. This size will obviously vary depending upon the size of the projectile chosen for insertion into the tube. Preferably, the aperture is between about 10 mm and about 25 mm in diameter. In a specific embodiment, the aperture is between about 10 mm and about 30 mm, and preferably about 15 mm, in length measured in a direction that is parallel to the length of the tube. In this specific embodiment, the aperture is between about 10 mm and about 30 mm, and preferably about 12 mm, in length measured in a direction that is perpendicular to the length of the tube.

The aperture must be closable to seal the tube sufficient to capture the air pressure produced behind the projectile when a user exhales into the mouthpiece end of the tube. Suitable closures include outer or inner sleeves, doors, covers, and the like that function to cover and seal the aperture. The aperture cover may also contain projections, contact areas, dials or the like as desired to make the aperture easier for the user to open or close. In a preferred embodiment, the closure is a second tube slidably or screwingly engaged with the cylindrical tube described above. Such second tube may fit internal to the cylindrical tube described above (hereinafter referred to as an “aperture tube”) or may fit around the cylindrical tube and be configured to slide over the cylindrical and seal the aperature in the cylindrical tube (hereinafter referred to as a “sleeve tube”). The aperture tube may be slidably engaged internally to the tube. In practice, the user introduces one or more projectiles through the aperture in the tube of this preferred embodiment and closes the aperture by sliding the aperture tube forward or backward within the tube to cover the aperture, before blowing into the tube to expel the projectile. In one preferred embodiment, the aperture tube has an external diameter between about 0.1 mm to about 1 mm less than the internal diameter of the tube and is slidably engaged within the tube. In another preferred embodiment, the sleeve tube has an internal diameter between about 0.1 mm to about 1 mm greater than the external diameter of the tube and is slidably engaged around the outside of the cylindrical tube.

In one embodiment, the tube is of a sufficient length to extend beyond the end of the tube when slid into place to cover the aperture in the tube. In a particularly preferred embodiment, the aperture tube contains an aperture that is substantially similar in shape and size to the aperture in the tube. In this embodiment, the user slides the aperture tube within or around the tube to align the aperture in the aperture tube with the aperture in the tube—allowing placement of one or more projectiles within the inner-most tube. The tubes are then slid such that the two apertures are no longer aligned and the inner-most tube is sealed along its length to allow the projectile to be expelled when the user exhales into the mouthpiece end of the inner-most tube.

In another embodiment, the sleeve tube is of sufficient length to allow the user to slide the sleeve tube along the length of the cylindrical tube to expose the aperture in the cylindrical tube, allowing placement of a projectile in the cylindrical tube. The user then slides the sleeve tube to cover the aperture in the cylindrical tube, thereby sealing the aperture in the cylindrical tube to allow expulsion of the projectile by exhaling into the mouthpiece on the cylindrical tube.

The aperture tube may be composed of any material compatible with the material(s) composing the tube, including wood, plastic, polymer(s), laminates, resins, metals, combinations of these materials and the like. Preferably, the aperture tube is composed of clear or translucent material(s) such that the user can judge the placement and movement of a projectile placed in the tube or aperture tube. Most preferably, the aperture tube is composed of polycarbonate.

In a specific embodiment, the cylindrical tube does not contain an aperture. In this embodiment, an aperture tube is included which is slidably or screwingly engaged within the internal diameter of the tube. This aperture tube contains an aperture of sufficient size to allow a user to dispose one or more projectiles within the aperture tube. In use, the user positions the desired projectile within the aperture tube through the aperture and then positions the aperture tube within the cylindrical tube to seal the aperture in the aperture tube, before expelling the projectile(s).

In another specific embodiment, the tube contains an aperture that is closed with an aperture tube that is slidingly or screwingly engaged over the outer diameter of the tube. The aperture tube of this embodiment does not contain an aperture. Rather, the user places one or more projectiles within the aperture tube and then positions the outer aperture tube over the tube including the aperture in the tube to seal the tube, prior to expelling the projectile(s).

The mouthpiece end of the tube may be a shaped end of the tube or of an aperture tube or may be a separate piece that is shaped for comfortable contact with the lips of the user. A preferred mouthpiece is formed by crimping or shaping the end of the cylindrical tube into a flattened mouthpiece that is wider but more narrow in cross-section than the cylindrical tube. Preferably, the mouthpiece is between about 20 mm and about 50 mm in width and between about 5 mm and about 30 mm in height. In a specific embodiment, the mouthpiece is detachable from the tube making replacement and cleaning of the mouthpiece easy for the user. In another specific embodiment, the mouthpiece may contain a projection or attachment that includes a means of measuring or estimating the volume or pressure of air expelled by the user into the mouthpiece. Such a projection or attachment may include graduations or scales in addition to moving indicators to effectively display an air pressure or relative air pressure expelled by the user into the mouthpiece. In another specific embodiment, the mouthpiece may contain a projection or attachment that includes a means of adjusting the resistance that must be overcome by the user to expel a projectile from the cylindrical tube. Such a projection may include a dial or slidable adjustment knob that allows the user to measure the resistance or relative resistance required to expel a projectile from the tube. In a preferred embodiment, the means of adjusting the resistance required to expel a projectile from the cylindrical tube may be locked or fixed in place over repeated uses of the apparatus to expel projectiles from the cylindrical tube.

The cylindrical tube and/or the aperture tube may contain projections suitable for different purposes. For example, the cylindrical tube and/or aperture tube may contain one or more internal projections that serve to guide a projectile in a specific direction or to increase the resistance that a user must exert to expel a projectile from the inner-most tube. Alternatively, the outer-most tube may contain projections designed to act as handles or to aid the user in inserting one or more projectiles into the tube. The outer-most tube may also contain projections that aid the user in directing projections expelled from the tube. In an embodiment containing both a cylindrical tube and either an aperture tube or a sleeve tube, either tube may contain a projection that serves as a catch or lock preventing the two tubes from being completely separated or disengaged. In a similar embodiment, either of the tubes may contain a projection that keeps the tubes from being slid or screwed apart beyond a point that is predetermined by the user or manufacturer of the apparatus.

In a specific embodiment, the tube may contain a projection or attachment that includes a means of measuring or estimating the volume or pressure of air expelled by the user into the cylindrical tube. Such a projection or attachment may include graduations or scales in addition to moving indicators to effectively display an air pressure or relative air pressure expelled by the user into the tube through the mouthpiece end of the tube.

In a specific embodiment, the tube may contain a projection or attachment that includes a means of adjusting the resistance that must be overcome by the user to expel a projectile from the tube. Such a projection may include a dial or slidable adjustment knob that allows the user to measure the resistance or relative resistance required to expel a projectile from the tube. In a preferred embodiment, the means of adjusting the resistance required to expel a projectile from the tube may be locked or fixed in place over repeated uses of the apparatus to expel projectiles from the tube.

In a specific embodiment, the apparatus may be formed from a single tube without an aperture tube. In this embodiment, the tube is formed with an aperture to allow the introduction of one or more projectiles into the tube. By covering the aperture, the user can then blow into the mouthpiece to expel the projectile from the tube. The aperture may be covered with an object to form a sufficiently-tight seal over the aperture to allow the projectile to be expelled from the tube. In a preferred embodiment, the user holds the tube in such a way to cover the aperture in the tube with the user's hand. In this embodiment, the aperture is formed in the tube in a way that makes it easy to both cover the aperture and hold the tube with one hand. Preferably, the aperture is formed in the tube such that a child may easily hold the tube and cover the aperture in the tube with one hand. It should be noted that the user is not required to form a perfectly air-tight seal over the aperture in order to expel the projectile from the tube by blowing through the mouthpiece. Instead, by covering most or all of the aperture in the tube, the user can expel a projectile from the tube by only partially covering the aperture, although greater air pressure must be exerted through the mouthpiece to expel the projectile from the tube if the aperture is not completely covered by the user than would be required to expel the same projectile from the tube if the aperture is completely covered.

The projectiles for use in the present apparatus may be any small object that can be effectively and routinely expelled from the tube using air pressure imparted from the lungs of the user through the mouthpiece end of the tube. For example, the projectile may be a ball, a marble, a rock, a bean bag, a pea, a small piece of paper, foam, plastic or food. Preferably, the projectile used by the user is a marshmallow and most preferably, the apparatus is configured for use with a projectile that is a “mini-marshmallow.”

Using the apparatuses of the present invention, the user may propel a projectile at someone or something by exerting sufficient air pressure through the tube. This action has the effect of challenging and building the user's respiratory system, including the respiratory musculature and lung capacity, through the repeated use of the apparatus. The user's repeated use is fostered and engaged, particularly amongst younger users, through the challenge and fun of hitting objects or people with the projectiles inserted into the tube. This effectively engages the user in a repetitive action and conditioning activity that may expand and strengthen the respiratory system while overcoming the repetitive and often dull activity of recurrent use of a traditional spirometer device. Additionally, the user, and particularly older users, may experience a sense of psychological satisfaction and relief in use of the apparatus of the present invention. Thus, the device has found use in anger management therapies in addition to the uses of strengthening the respiratory system and the immune system and as a toy as described above.

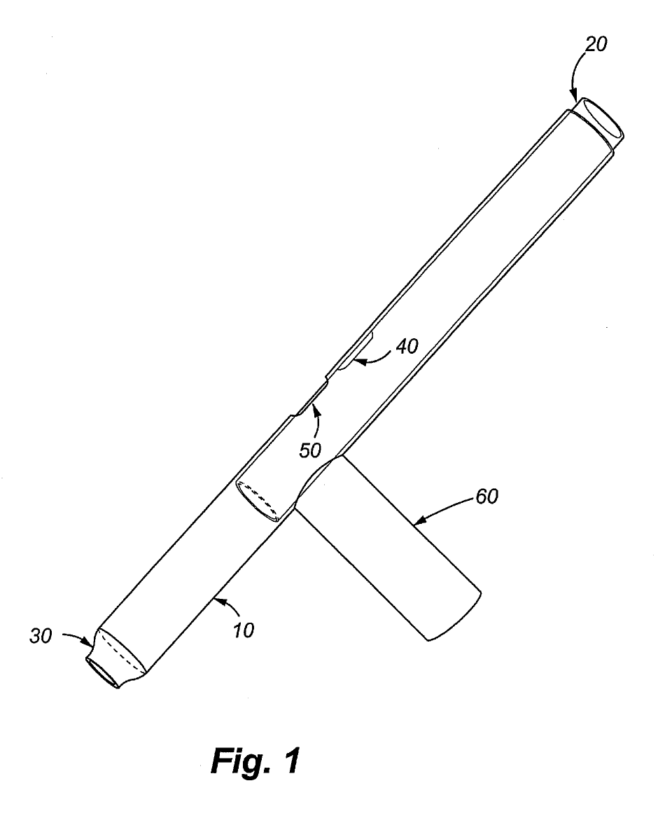

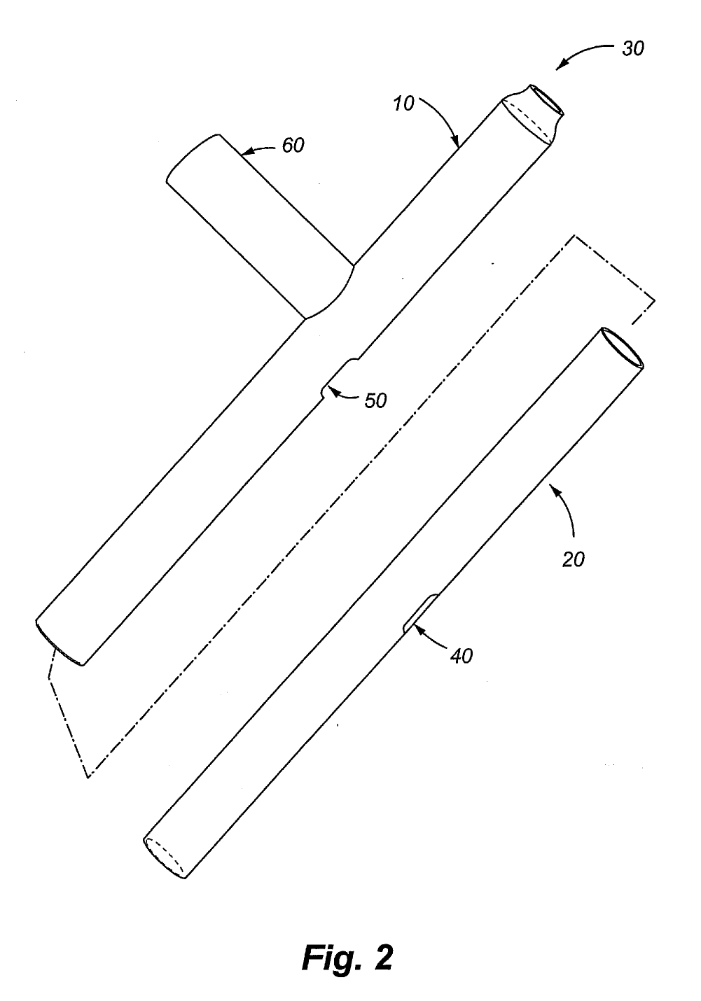

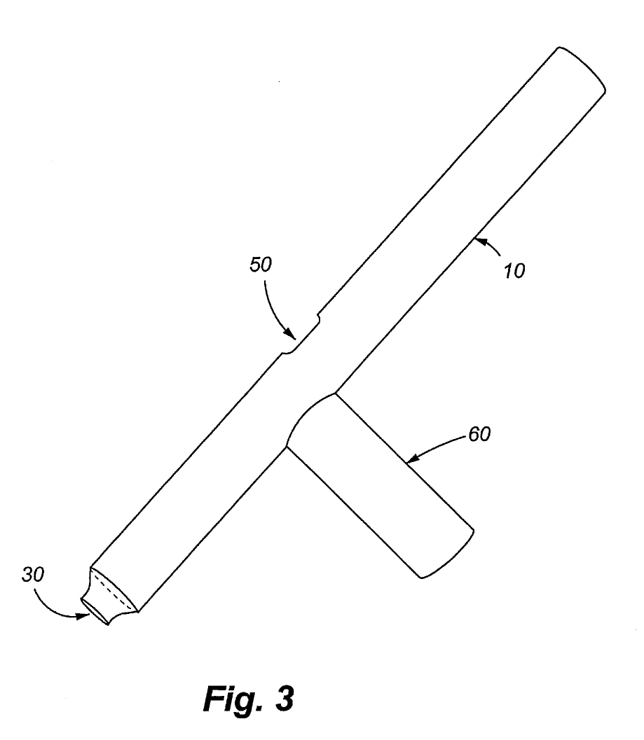

Referring to FIG. 1, a preferred embodiment of the present invention is shown wherein an aperture tube (20) is slidably engaged within a cylindrical tube (10) that has a single end crimped into a mouthpiece (30) and a projection (60) forming a handle. The cylindrical tube (10) has an aperture (50) that can be aligned with a similarly-sized aperture (40) in the aperture tube (20). FIG. 2 shows the same embodiment in which the aperture tube (20) has been removed from within the tube (10). The separate apertures (40 and 50) are more clearly visible in FIG. 2. FIG. 3 shows the same embodiment in which the projection (60) from the cylindrical tube (10) that functions as a handle for the user, can be clearly seen. The projection (60) is attached to the cylindrical tube (10) with glue capable of securely bonding the polycarbonate parts. Suitable adhesives include epoxy.

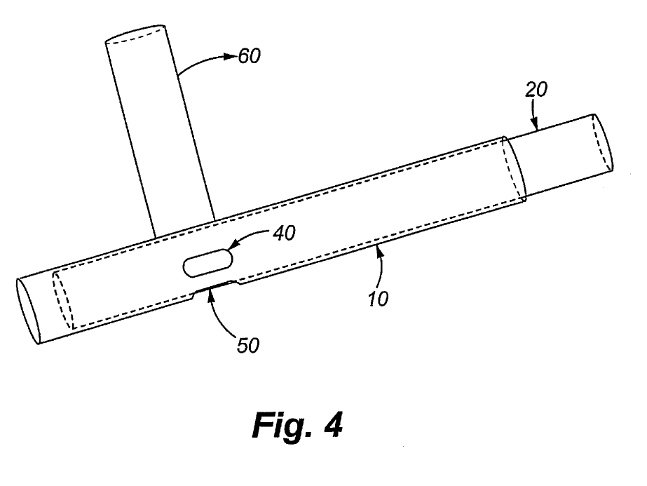

FIG. 4 shows the same embodiment in which the aperture tube (20) is slidably engaged within the tube (10) and the apertures (40 and 50) have been aligned length-wise along the tube (10) but have been offset around the circumference of the tube (10), thereby sealing the tube (10) and the aperture tube (20) such that a projectile placed within the internal aperture tube (20) can be expelled by exerting air pressure from the user's lungs through the mouthpiece (30).

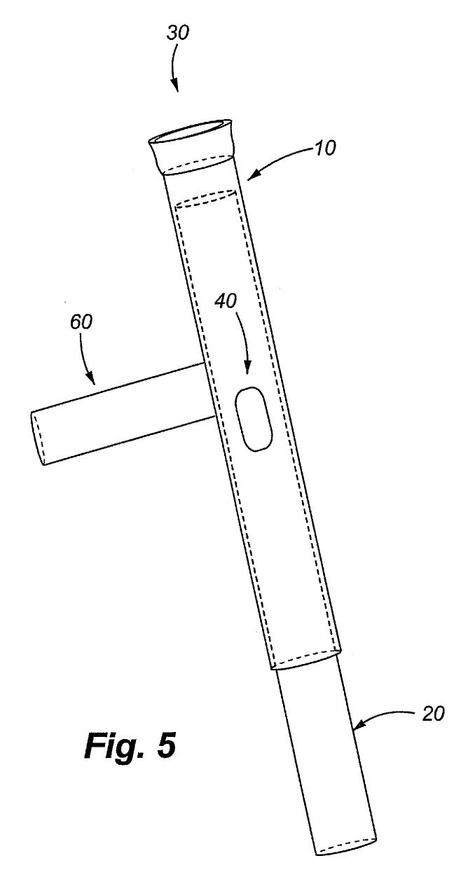

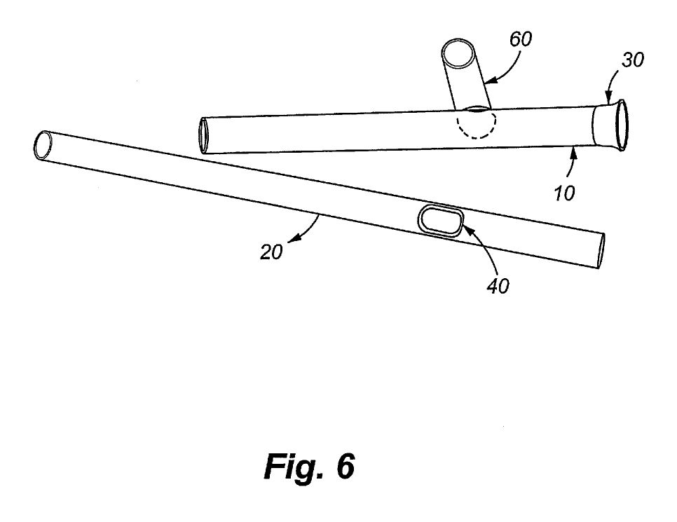

Referring to FIG. 5, another particularly preferred embodiment of the invention is shown wherein an aperture tube (20) is slidably engaged within a cylindrical tube (10) that has a single end crimped into a mouthpiece (30) and a projection (60) forming a handle. The aperture tube (20) has an aperture (40) suitable for the passage of projectiles to be displaced within the aperture tube. FIG. 6 shows the same preferred embodiment in which the aperture tube (20) has been removed from within the cylindrical tube (10).

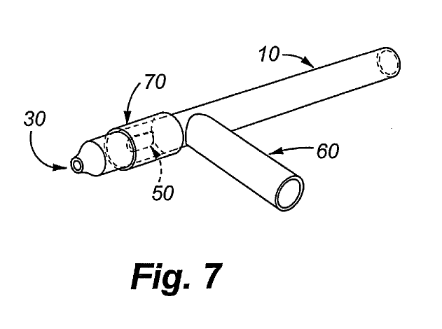

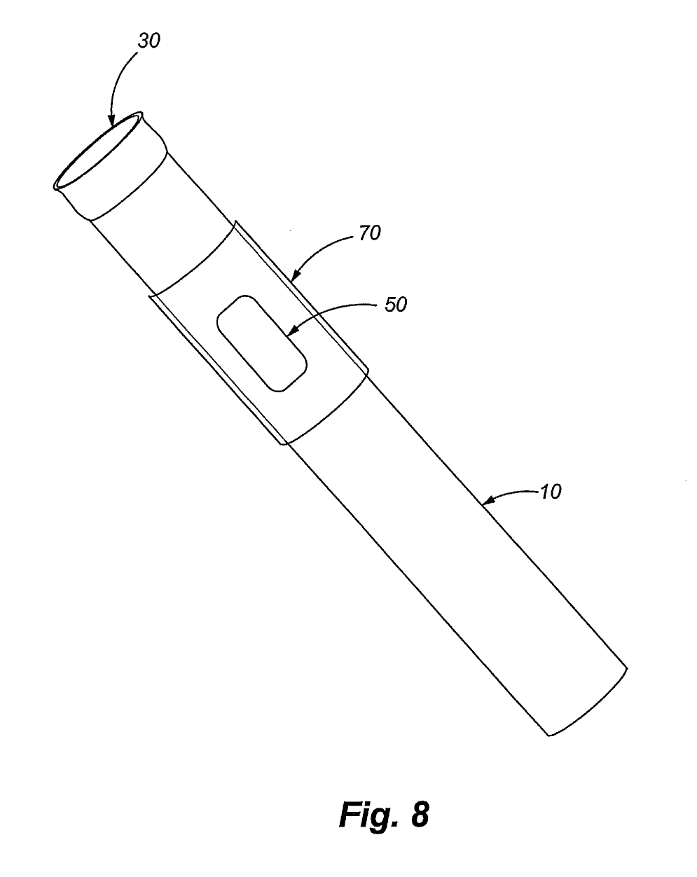

Referring to FIG. 7, a preferred embodiment of the present invention is shown in which a sleeve tube (70) is displaced around the cylindrical tube (10) to cover aperture (50). The sleeve tube (70) can be slid along the cylindrical tube towards the mouthpiece (30) to expose the aperture (50). Projectiles can then be displaced in the cylindrical tube (10) through the aperture (50), and the sleeve tube (70) can be slid over the aperture (50) to seal the cylindrical tube (10). In the embodiment shown in FIG. 7, the sleeve tube (70) is displaced over the cylindrical tube (10) between the mouthpiece (30) and a handle (60). The mouthpiece (30) is wider than the inner diameter of the sleeve tube (70) such that the sleeve tube (70) cannot slide off of the cylindrical tube (10) as it is trapped between the handle (60) and the mouthpiece (30). This is seen best in FIG. 8, which is a top view of the embodiment shown in FIG. 7. From this view, it can be seen that the mouthpiece (30) is wider than the inner diameter of the sleeve tube (70) such that the sleeve tube (70) cannot slide off of the cylindrical tube (10).

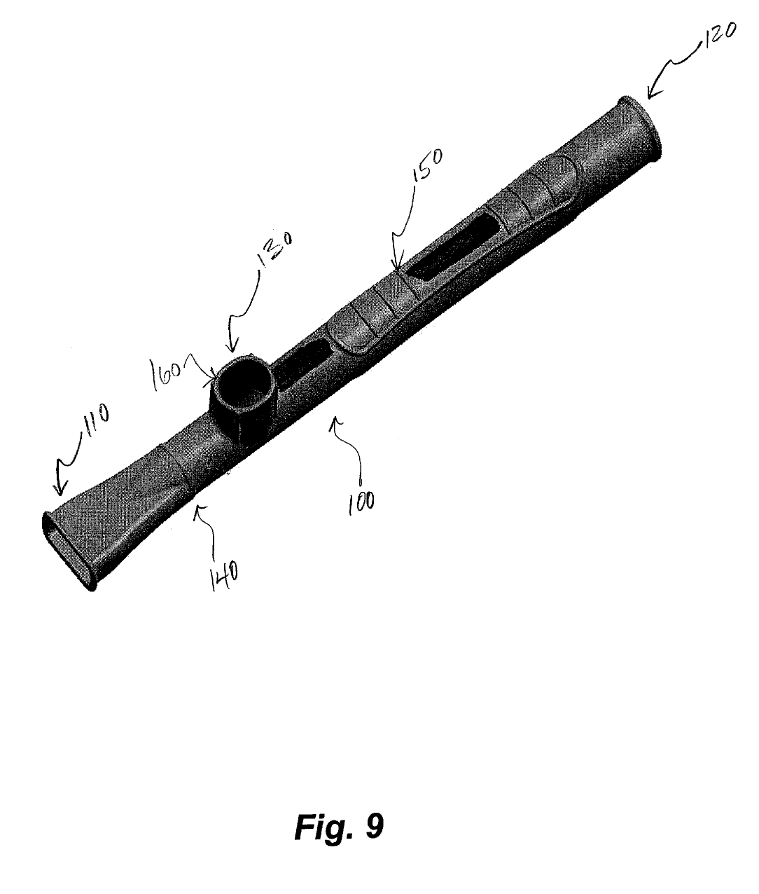

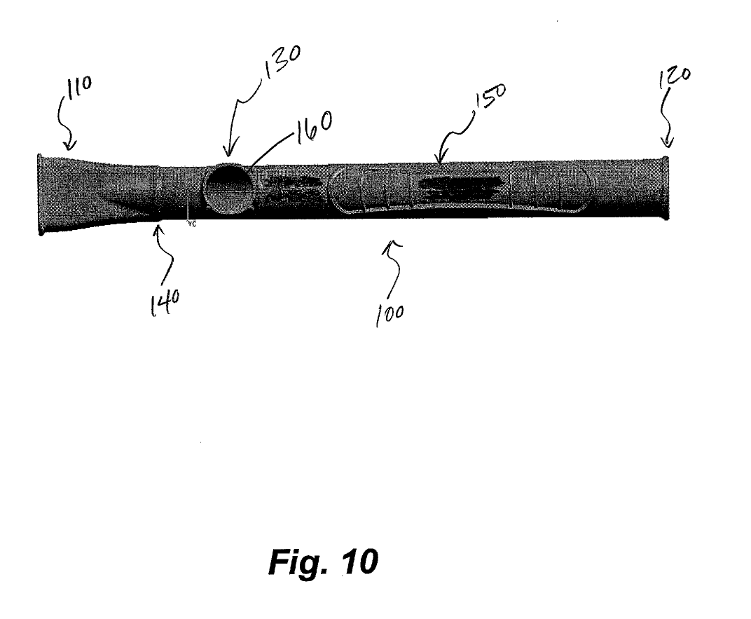

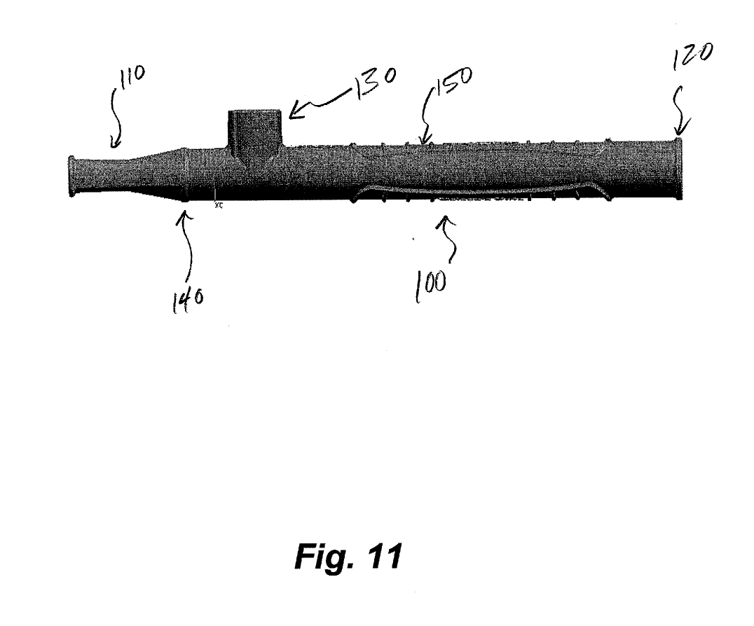

Referring to FIGS. 9-12, one embodiment of the present invention is an apparatus (100) composed of a tube (150) having a first end formed into a mouthpiece (110) and an opposite end (120). The tube has an open aperture (160) of a size sufficient to allow the introduction of a projectile into the tube (150) through the aperture (160). The aperture may be formed in the tube along the length of the tube (150) between the mouthpiece (110) and the opposite end (120) of the tube (150).

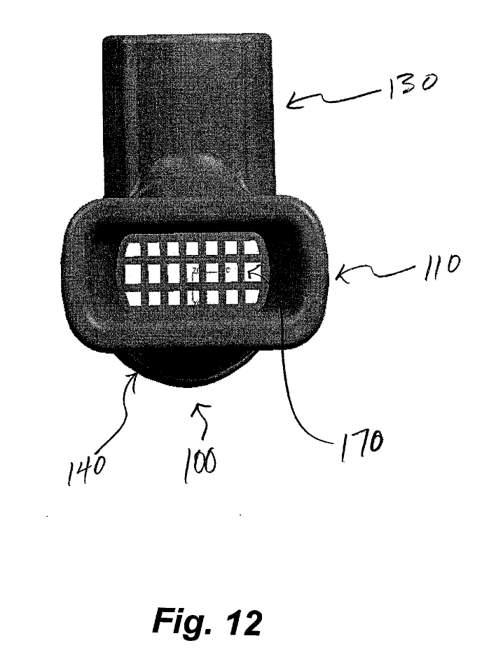

The mouthpiece (110) may be a formed end of the tube (150) or may be a produced as a separate piece that screwingly or slidingly engages in one end of the cylindrical tube at a position (140) between the first end of the tube and the aperture 160. In a preferred embodiment depicted in FIG. 12, the mouthpiece (110) has a rectangular shape that can comfortably be placed in the mouth or against the lips of the user. Preferably, the mouthpiece has a screen (170) positioned within the interior circumference of the mouthpiece to prevent objects of a size greater than the openings in the screen from moving from within the tube (150) out of the mouthpiece (110). The screen (170) may be formed as a separate piece that fits tightly or is affixed within the mouthpiece (110) or the screen (170) may be formed as an integral part of the mouthpiece (110). The screen may formed with varying sizes of holes in the screen. The smaller the holes in the screen (170), the greater is the air pressure required to be introduced into the tube (150) through the mouthpiece (110) in order to expel a projectile from within the tube (150) through the opposite end (120) of the tube (150).

The aperture (160) may take any form that will allow the introduction of a projectile into the interior of the tube. In a preferred embodiment, the aperture (160) is formed in a chimney (130) projecting in a direction substantially perpendicular from the tube (150). The chimney (130) is preferably circular and of a height that allows a user with small hands to hold the tube (150) in one hand while simultaneously covering the aperture (160) in the tube (150) by covering all or part of the chimney (130) with the same hand. Preferably, the chimney (130) is between about 10 mm and about 80 mm in height as measured from the interior surface of the tube (150) opposite the aperture (160) to the “top” or end of the chimney opposite the tube (150).

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. An apparatus comprising a tube having a mouthpiece on one end and at least one aperture along the length of the tube.

2. The apparatus of claim 1, wherein the tube is a circular tube.

3. The apparatus of claim 1, wherein the tube has a length between about 4 inches and about 36 inches.

4. The apparatus of claim 1, wherein the tube has a diameter between about 5 mm and about 20 mm.

5. The apparatus of claim 1, wherein the tube is composed of a material selected from the group consisting of wood, plastic, polymer, laminate, resin, metal, polycarbonate and combinations thereof.

6. The apparatus of claim 1, wherein the aperture is chimney formed as a substantially perpendicular projection from the tube having an open end opposite the junction with the tube and adapted to receive a projectile through the open end.

7. The apparatus of claim 1, further comprising a closure for the aperture selected from the group consisting of a sleeve, a door and an aperture tube.

8. The apparatus of claim 1, wherein the mouthpiece comprises a crimped end of the tube.

9. The apparatus of claim 1, wherein the mouthpiece comprises a material that is removably-engaged from the tube.

10. The apparatus of claim 1, wherein the mouthpiece comprises a screen positioned within the mouthpiece to prevent an object from moving from within the tube out the end of the tube trough the mouthpiece.

11. A kit comprising the apparatus of claim 1 and at least one projectile selected from the group consisting of a marshmallow, a mini-marshmallow, a pea, a ball and a piece of foam.

12. A method of enhancing the respiratory system in a user comprising inserting a projectile through the aperture and into the tube of the apparatus of claim 1, placing the lips of the user on the mouthpiece of the apparatus, and exhaling through the mouthpiece with sufficient force to expel the projectile from the tube.
